(19) United States
(12) Patent Application Publication
(10) Pub. No.: US 2007/0178107 A1
(43) Pub. Date: Aug. 2, 2007

(54) Method and means for treating solid tumors

(76) Inventor: Essam T. Awdalla, Morrisville, NC (US)

Correspondence Address: Essam T. Awdalla, 124 Black Ridge Street, Morrisville, NC, 27560, US

(21) Appl. No.: 11/595,291

(22) Filed: Nov. 10, 2006

Related U.S. Application Data
(63) Continuation-in-part of application No. 11/399,281, filed on Apr. 6, 2006.
Application No. 11/399,281 is a continuation-in-part of application No. 11/343,694, filed on Jan. 31, 2006.

(51) Int. Cl. A61K 39/395; A61K 48/00; A61K 31/7048; A61K 31/704; A61K 31/57; A61K 31/4745; A61K 31/366

(52) U.S. Cl. 424/155.1; 424/178.1; 514/731; 514/54; 514/56; 514/44; 514/12; 514/393; 514/171; 514/34; 514/283; 514/27; 514/453

(57) ABSTRACT

The present invention relates to method and means for treating a solid tumor using a number of, in vitro prepared, anticellular agent(s)-carrying blood platelets to induce a thrombus formation within the tumor vasculature, and at the same time to deliver a high concentration of an anticellular agent within the tumor. The blood platelets are targeted and attached to the tumor vasculature using in vivo assembled binding complexes, each having at least one binding site specifically binding to tumor cells or to tumor-associated vasculature, and at least one binding site specifically binding to a blood platelet surface. The platelet-mediated thrombus formed within the tumor vasculature leads to occlusion of the tumor vasculature, with ultimate destruction of the centrally located tumor cells. This is followed by destruction or impairing the growth or cell division of the peripherally located tumor cells, by the anticellular agent(s) carried by the blood platelets.

### CROSS-REFERENCE TO RELATED APPLICATION(S)

This non-provisional utility patent application claims the benefit of one prior filed co-pending non-provisional patent application; the present application is a continuation-in-part of U.S. patent application Ser. No. 11/399,281, filed Apr. 6, 2006, which is a continuation-in-part of U.S. patent application Ser. No. 11/343,694, filed Jan. 31, 2006, which are incorporated herein by reference in their entirety.

### FIELD OF THE INVENTION

The present invention relates generally to a method for treating solid tumors in a mammal, and more particularly to a method for treating a vascularized solid tumor by targeting a plurality of anticellular agent-carrying blood platelets to the vasculature of the tumor, to induce a thrombus formation within the tumor vasculature, and at the same time to deliver a high concentration of the anticellular agent to the tumor cells, and thus, clearing solid tumors entirely from the mammalian body.

### BACKGROUND OF THE INVENTION

Cancer represents a group of diseases characterized by uncontrolled growth and proliferation of abnormal cells, which, if not controlled, results in death of the host. Cancer continues to be one of the most serious diseases threatening human and animal health and life. The American Cancer Society estimated that about 1,372,910 new cancer cases were diagnosed in the USA in 2005, with solid tumor cases accounting for more than 90% of all cancer cases. In the same year, 570,280 cancer patients were expected to die in the USA. These statistics translate to more than 1600 deaths per day. Cancer is the second leading cause of death in the USA next to the coronary heart diseases.

The traditional treatment of cancer patients involves a combination of surgery, radiotherapy and/or chemotherapy, unfortunately, combined treatment with all three modalities have not shown to be effective against all cancer and tumor cells, due to the wide heterogeneity of cancer cells regarding their metabolism, enzyme composition, growth rate and gene errors, with some of the cancer cells being usually resistant to each of the used treatment modalities. The resistant cells survive, seed, and continue to grow in the living host, with subsequent treatments being less effective at killing the cancer cells.

As a solid tumor grows, in order to sustain itself, it must develop its own blood supply. This blood supply, however, is much different from the blood supply to normal tissues. The blood vessels formed in tumors are typically highly irregular and tortuous. They may have arterio-venous shunts and blind ends, and lack smooth muscle or nerves and have incomplete endothelial linings and basement membranes. This leads to low overall levels of oxygen in most tumors. Many tumors have areas of extreme hypoxia. (Brown, J. M. “Exploiting the hypoxic cancer cell: mechanisms and therapeutic strategies” Molecular Medicine Today, April 2000 (Vol. 6)). Such hypoxic areas are known to be refractory towards many of the currently available treatments for solid tumor cancers, including radiation therapy and chemotherapy.

Several unconventional approaches for treating solid tumors are being proposed continuously, with targeting the tumor vasculature with Vascular disrupting agents (VDAs) being the most promising of these approaches. Vascular disrupting agents (VDAs) are designed to cause a rapid and selective shutdown of the blood vessels of tumors. Unlike antiangiogenic drugs that inhibit the formation of new vessels, VDAs occlude the pre-existing blood vessels of tumors to cause tumor cell death from ischemia and extensive hemorrhagic necrosis. There are broadly two types of VDAs, small molecules and ligand-based, which are grouped together, because they both cause acute vascular shutdown in tumors leading to massive tumor necrosis. The small molecule VDAs include the microtubulin destabilizing drugs, combretastatin A-4 disodium phosphate, ZD6126, AVE8062, and Oxi 4503, and the flavonoid, DMXAA. Ligand-based VDAs use antibodies, peptides, or growth factors that bind selectively to tumor blood vessels to target tumors with agents that occlude blood vessels. The ligand-based VDAs include fusion proteins (e.g., vascular endothelial growth factor linked to the plant toxin gelonin), immunotoxins (e.g., monoclonal antibodies to endoglin conjugated to ricin A), antibodies linked to cytokines, liposomally encapsulated drugs, and gene therapy approaches.

However, as VDAs are designed to occlude the tumor vasculature, so they cut off the blood supply to only the centrally located tumor cells, which rely on the tumor vasculature for their nutrition, leading to their destruction, while sparing the peripherally located outer rim of tumor cells, which rely on the surrounding blood vessels and interstitial fluids for their nutrition. The surviving outer rim of tumor cells will eventually grow and replace the destroyed central part of the tumor. Thus there is a need for another VDAs-like approach for treating solid tumors, with which both the centrally located and the peripherally located tumor cells may be simultaneously destroyed.

Prior art documents include components related to the field of the present invention but lack an integrated, combined solution that is provided by the present invention, including the following:

Targeting antibodies carrying diagnostic or therapeutic agents to the vasculature of solid tumor masses, through recognition of tumor vasculature-associated antigens described by Thorpe et al in U.S. Pat. Nos. 5,776,427, 5,863,538;
delivering a compound of interest to a thrombogenic surface, using fixed-dried blood platelets carrying said compound, as described by Nichols, Timothy C.; et al. in U.S. patent application Ser. No. 11/149,515;
monoclonal antibodies and their fragments, which may be derived from any species (including humans) or may be formed as chimeric proteins which employ sequences from more than one species, using conventional techniques, such as hybridoma synthesis, recombinant DNA techniques and protein synthesis. See, generally, Kohler and Milstein, Nature, 256: 495-97, 1975; and Eur. J. Immunol., 6: 511-19, 1976; both of which are incorporated herein by reference;
human, or humanized, monoclonal antibodies recognizing surface antigens of cancer cells. Non limiting examples are described by Hosokawa, et al. in U.S. Pat. No. 6,787,153, by Taniguchi, et al. in U.S. Pat. No. 4,800,155, by Abe, et al. in U.S. Pat. No. 5,024,946, by Hagiwara, et al. in U.S. Pat. No. 5,093,261, and by Anderson, et al. in U.S. Pat. No. 6,753,420 and U.S. Pat. No. 6,417,337; all of which are incorporated herein by reference;
antibodies recognizing tumor associated antigens. Non limiting examples includes antibodies targeting tumor vasculature (Duijvestijn et al., J. Immunol., 138(3):713-719, 1987; Hagemeier et al., Int. J. Cancer, 38:481-488, 1986; Bruland et al., Int. J. Cancer, 38:27-31, 1986; Murray et al., Radiotherapy and Oncology, 16:221-234, 1989; and Schlingemann et al., Laboratory Investigation, 52(1):71-76, 1985), and antibodies targeting tenascin, a large molecular weight extracellular glycoprotein expressed in the stroma of various benign and malignant tumors (Shrestha et. al., Eur. J. Cancer B. Oral. Oncol., 30B(6):393-9, 1994; and Tuominen and Kallioinen, J. Cutan. Pathol. 21(5):424-9, 1994.), all of which are incorporated herein by reference;
anti-platelet antibodies described by Gralnick in U.S. Pat. No. 5,366,865, which is incorporated herein by reference; and
the use of streptavidin, avidin, and biotin molecules to conjugate molecules to one another, to form biotinylated protein molecules, biotinylated protein-avidin or avidin like complexes, or multicomponent conjugates, both in vitro and in vivo, is well known in the Art. See, generally, P. Webber et al., “Science, vol. 243, pp. 85-88, Jan. 6, 1989”, M. Wilchek et al, “Analytical Biochemistry, vol. 171 pp. 1-32, 1988”, Otto C. Boerman et al., “Pretargeted Radioimmunotherapy of Cancer: Progress Step by Step”, Journal of Nuclear Medicine Vol. 44 No. 3 400-411, Bayer et al., “Trends in Biochemical Science, 3, N257, November 1978”, and Paganelli G, Riva P, Deleide G, et al. “Int J Cancer Suppl. 1988; 2: 121-125”, all of which are incorporated herein by reference.

However, non of these references suggest targeting anticellular agent-carrying blood platelets to the vasculature of a solid tumor, and thus inducing the formation of a thrombus within the tumor vasculature and, at the same time, delivering a high concentration of an anticellular agent to the tumor cells, and thus forming a platelet-mediated thrombus within the tumor vasculature leading to occlusion of the tumor vasculature, with ultimate destruction of the centrally located tumor cells, followed by destruction or impairment of the growth or cell division of the peripherally located tumor cells by the anticellular agent carried by the blood platelets and concentrated within the tumor.

### SUMMARY OF THE INVENTION

The present invention is directed to and provides, in one aspect of the invention, a method for treating a vascularized solid tumor using a plurality of anticellular agent-carrying blood platelets targeted to the vasculature of the tumor, to induce a thrombus formation within the tumor vasculature and, at the same time, to deliver a high concentration of the anticellular agent to the peripherally located tumor cells, and thus, clearing solid tumors entirely from a mammalian body.

The present invention is further directed, in another aspect of the invention, to means for targeting a plurality of anticellular agent-carrying blood platelets to a tumor vasculature, to induce a thrombus formation within the tumor vasculature and, at the same time, to deliver a high concentration of the anticellular agent to the peripherally located tumor cells, while avoiding any deterioration in the functions of the spleen or any other vital body organ.

The present invention is further directed, in another aspect of the invention, to a schedule for treating a mammal affected by multi-focal secondary metastases deposits of a solid tumor, with at least one of the secondary metastases deposits being vascularized, and with at least another one of the secondary metastases deposits being not-yet vascularized.

As used herein, the term “parenteral administration” refers to and includes any route through which a compound is administered to a mammal other than through the digestive tract, non limiting examples of such routes include: intravenous injection, intra-arterial injection, intracavitary injection, intramuscular injection, and injection through an intravenous line, cannula, catheter, or the like; the term “parenteral administration of a sub-therapeutic dose of a small molecular Vascular Disrupting Agent (VDA)” refers to and includes the administration of any small molecular Vascular Disrupting Agent (VDA), to a mammal, at any dose smaller than its minimal therapeutically effective dose, with the minimal therapeutically effective dose of a VDA being the dose at or above which the administration of the VDA to the mammal will result in acute irreversible occlusion of any tumor vasculature; the term “anti-tumor binding component” refers to and includes any compound having a binding region specifically binding to an antigen or a receptor present on the outer surface of a tumor cell, or present on the outer surface of a component of a tumor associated vasculature or stroma; the terms “first ligand, second ligand, and anti-ligand” refers to and includes any complementary set of molecules that specifically bind to each other; the term “anti-platelet binding component” refers to and includes any compound having a binding region specifically binding to an antigen or a receptor present on the outer surface of a blood platelet; the term “carrying” refers to and includes either containing the anticellular agent within the blood platelets, attaching the anticellular agent to the outer surface of the blood platelet, or both; the term “anticellular agent” refers to and includes any agent that destroys, impair the growth or cell division, or irreversibly alter the metabolism of a cancer cell; the term “vascularized secondary metastases deposit of a solid tumor” refers to and includes any secondary metastases deposit of a solid tumor having its own feeding blood vasculature; and the term “not-yet vascularized secondary metastases deposit of a solid tumor” refers to and includes any secondary metastases deposit of a solid tumor which did not develop its own feeding blood vasculature yet.

Typical vascularized solid tumors are solid tumors which require a vascular component for the provision of oxygen and nutrients. Exemplary solid tumors to which the present invention is directed, include, but are not limited to, carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, neuroblastomas, different types of sarcomas, and the like.

Accordingly, the present invention provides method and means for treating a mammal from a vascularized solid tumor using a number of, in vitro prepared, anticellular agent-carrying blood platelets to induce a thrombus formation within the tumor vasculature, and at the same time to deliver a high concentration of the anticellular agent to the peripherally located tumor cells.

In the method provided in the present invention, a sub-therapeutic dose of a small molecular Vascular Disrupting Agent (VDA) is used to selectively disrupt the endothelial lining of the tumor vasculature, while maintaining adequate rate of blood flow within the tumor vasculature. Although small molecular VDAs are tested and developed to provide acute irreversible occlusion of the tumor vasculature, yet conducted preclinical studies showed that the administration of a low sub-therapeutic dose of a small molecular VDA results in disruption of the endothelial lining of the tumor vasculature, with initial marked reduction in the tumor blood flow, followed by near complete recovery of the blood flow within the tumor vasculature after 24 hours. A non limiting example of these studies is disclosed by Prise V E, Honess D J, Stratford M R L, Wilson J, Tozer G M., in “The vascular response of tumor and normal tissues in the rat to the vascular targeting agent, combretastatin A-4-phosphate, at clinically relevant doses”. (Int J Oncol 2002; 21:717-26.). This study is incorporated herein by reference.

In a preferred embodiment of the present invention, the provided method for treating a vascularized solid tumor includes the steps of:
a) parenteral administration of a sub-therapeutic dose of at least one small molecular Vascular Disrupting Agent (VDA), to disrupt the endothelial lining of the tumor vasculature, while maintaining adequate rate of blood flow within the tumor vasculature, and thus, directly exposing the underlying tumor cells to the circulating blood;
b) Targeting a number of, in vitro prepared, anticellular agent carrying blood platelets to the tumor cells exposed in step (a), leading to immobilization of the said anticellular agent-carrying blood platelets within the tumor vasculature;
c) allowing for the induction of a thrombus formation within the tumor vasculature, leading to occlusion of the tumor vasculature and destruction of the centrally located tumor cells, followed by rupture of the said anticellular agent-carrying blood platelets, with release of their anticellular agent content; and
d) arranging for the delivery of the anticellular agent released from the ruptured blood platelets in step (c), to the peripherally located tumor cells.

In another preferred embodiment of the present invention, the provided method for treating a vascularized solid tumor includes the steps of:
a) parenteral administration of a sub-therapeutic dose of at least one small molecular Vascular Disrupting Agent (VDA), to disrupt the endothelial lining of the tumor vasculature, while maintaining adequate rate of blood flow within the tumor vasculature, and thus directly exposing the underlying tumor cells to the circulating blood. In a preferred embodiment, this step is preceded and/or accompanied by the administration of at least one anti-coagulant agent, e.g. Heparin, to prevent the formation of any small thrombi within the tumor vasculature, which is known to accompany the administration of VDA;
b) parenteral administration of a number of at least one type of anti-tumor binding component—first ligand complexes, which will attach themselves to the exposed tumor cells. In a preferred embodiment, this step is also preceded and/or accompanied by the administration of at least one anti-coagulant agent, e.g. Heparin, to prevent the formation of any small thrombi within the tumor vasculature;
c) parenteral administration of a number of anti-ligands, which will attach themselves to the anti-tumor binding component—first ligand complexes, already attached to the tumor cells. In a preferred embodiment, this step is also preceded and/or accompanied by the administration of at least one anti-coagulant agent, e.g. Heparin, to prevent the formation of any small thrombi within the tumor vasculature;
d) parenteral administration of a number of, in vitro prepared, blood platelets, each blood platelet carrying at least one anticellular agent and having at least one anti-platelet binding component—second ligand complex attached to its outer surface. In a preferred embodiment, this step is also preceded and/or accompanied by the administration of at least one anti-coagulant agent, e.g. Heparin, to delay the onset of the self-induced thrombus formation within the tumor vasculature, as will be described herein after;
e) allowing the blood platelets to link to the tumor cells through in vivo formation of anti-tumor binding component—first ligand—anti-ligand—second ligand—anti-platelet binding component complexes;
f) parenteral administration of a number of anti-ligands to allow more anticellular agent-carrying blood platelets to link to the blood platelets already linked to the tumor cells, through in vivo formation of anti-platelet binding component—second ligand—anti-ligand—second ligand—anti-platelet binding component complexes. In a preferred embodiment, this step is also preceded and/or accompanied by the administration of at least one anti-coagulant agent, e.g. Heparin, to delay the onset of the self-induced thrombus formation within the tumor vasculature, as will be described herein after, until most of the administered anticellular agent-carrying blood platelets are immobilized within the tumor vasculature;
g) allowing for the initiation of the self-induced thrombus formation within the tumor vasculature, as will be described herein after, leading to occlusion of the tumor vasculature and destruction of the centrally located tumor cells, followed by rupture of the anticellular agent-carrying blood platelets included within the formed blood thrombus, with release of their anticellular agent content; and
h) encouraging the mammal to exercise few times a day, for several days, to evenly disperse the anticellular agent released from the ruptured blood platelets within the debris of the centrally located tumor cells, which enables delivering the anticellular agent to the peripherally located tumor cells, as will be described herein after.

In a preferred embodiment, the formation of a blood thrombus within the tumor vasculature in step (g) is followed by removal of the freely circulating residual portion of the administered anticellular agent-carrying blood platelets, which were not included within the thrombus formed within the tumor vasculature, from the mammal's blood stream.

Non limiting examples of small molecular Vascular Disrupting Agent (VDA) for use with the present invention include the microtubulin destabilizing drugs, combretastatin A-4 disodium phosphate, ZD6126, AVE8062, and Oxi 4503; and the flavonoid, DMXAA.

Any compound having a binding region specifically binding to an antigen or a receptor present on the outer surface of a tumor cell, or present on the outer surface of a component of a tumor associated vasculature or stroma can be used as the anti-tumor binding component, according to the present invention. In a preferred embodiment of the present invention, at least two types of anti-tumor binding components are used, with one of them specifically binding to tumor cells, and the other one specifically binding to a tumor associated vasculature or stroma. Any complementary set of molecules that specifically bind to each other can be used as the first ligand, second ligand, and anti-ligand according to the present invention. Also, any compound having a binding region specifically binding to an antigen or a receptor present on the outer surface of a blood platelet can be used as the anti-platelet binding component according to the present invention.

“Platelets” utilized in carrying out the present invention are, in general, of animal, and preferably mammalian, origin (e.g., pig, sheep, cow, horse, goat, cat, dog, mouse, rat, human, etc.). Platelets may be derived from the same species into which the platelets are introduced, or from a species different from the species into which the platelets are introduced. Either freshly isolated platelets or rehydrated fixed-dried platelets can be used with the present invention.

Any agent that destroys, impairs the growth or cell division, or irreversibly alters the metabolism of cancer cells can be used as the anticellular agent, according to the present invention. In a preferred embodiment of the present invention, the anticellular agent is contained within the blood platelets. In another preferred embodiment, the anticellular agent is attached to the outer surface of the blood platelet. In yet another preferred embodiment, more than one anticellular agent are used, with at least one anticellular agent being contained within the blood platelet, and at least one anticellular agent being attached to the outer surface of the blood platelet.

In general, the anticellular agent(s) to be delivered is coupled to or associated with the platelets so that each platelet carries, or has associated therewith, at least 1,000, and more preferably at least 10,000, individual molecules of the agent to be delivered.

As the life span of the platelets within the formed thrombus is approximately 10 days, so, everyday about 10% of the platelets attached to the cancer cells, or to the tumor associated vasculature or stroma, will rupture spontaneously. The ruptured platelets will release ADP (Adenosine diphosphate), thromboxane A2, serotonin, phospholipids, lipoproteins, and other proteins, leading to the activation of the nearby blood platelets and the initiation of a blood coagulation cascade. See, for example: Hechler, B., Leon, C., Vial, C., Vigne, P., Frelin, C., Cazenave, J. P., and Gachet, C. (1998) Blood 92, 152-159. The activation of the blood platelets modifies their membranes in such a way to allow fibrinogen to adhere to them, which results in attaching the fibrinogen net of the formed blood thrombus to the outer surface of the activated blood platelets. And thus, the formed blood thrombus will be indirectly attached to the tumor cells.

The formed blood thrombus occludes the blood vessels in-between the cancer cells, and thus, cutting off the blood supply to the centrally located cancer cells, leading to their destruction. This is followed by rupture of the platelets immobilized at the periphery of the formed blood thrombus, with release of their anticellular agent content. Then, the patient is encouraged to exercise few times a day, for several days, to mechanically agitate the cellular debris within the central part of the tumor, leading to even dispersion of the anticellular agent, released from the ruptured blood platelets, within the debris of the centrally located tumor cells, and thus enabling the anticellular agent to reach to the intact peripherally located tumor cells at high concentration, leading to their subsequent destruction; or impairment of their growth or cellular division; or irreversibly altering their metabolism, according to the type of the anticellular agent(s) used, and thus, all the cells of the solid tumor are destroyed.

The freely circulating residual portion of the administered anticellular agent-carrying blood platelets, which were not included within the thrombus formed within the tumor vasculature, is either selectively, or non-selectively removed from the mammal's blood stream, to avoid delivering a high concentration of the used anticellular agent(s) to the spleen, which will markedly deteriorate the splenic ability to function properly afterwards.

In a preferred embodiment of the present invention, the provided method is preceded and/or accompanied and/or followed by conventional enteral or parenteral administration of a therapeutic dose of at least one anticellular agent, to destroy any small sized non vascularized solid tumors present within the mammal, as well as early implanted and not-yet implanted tumor metastases.

Also, in a preferred embodiment of the present invention, the provided method is preceded and/or accompanied by the administration of at least one immuno-suppressive agent to the mammal, to safe guard against the development of an immune response against the administered components which will hinder their re-administration in a following setting, if needed.

The present invention is further directed, in another aspect of the invention, to a schedule for treating a mammal affected by multi-focal, variable-sized, secondary metastases deposits of a solid tumor, with at least one of the secondary metastases deposits being vascularized, and with at least another one of the secondary metastases deposits being not-yet vascularized.

In a preferred embodiment, the provided schedule for treating the mammal affected by said multi-focal, variable-sized, secondary metastases deposits of the solid tumor includes the steps of:
a) treating the mammal using the method provided in the present invention for treating a mammal from a vascularized solid tumor, and described in full details herein before, to destroy the vascularized secondary metastases deposits of the tumor;
b) administration of at least one agent to prevent/minimize the implantation of new tumor secondary metastases deposits within the mammal's body, for a period of time sufficient for the not-yet vascularized secondary metastases deposits of the tumor to grow and develop its own vasculature; and
c) re-treating the mammal using the method provided in the present invention for treating a mammal from a vascularized solid tumor, to destroy the now vascularized secondary metastases deposit(s) of the tumor, and thus, completely clearing the secondary metastases deposits of the tumor from the mammal's body.

In a preferred embodiment, the agent(s) administered to prevent/minimize the implantation of new tumor secondary metastases deposits within the mammal's body is selected from the group consisting of anti-coagulants; platelet inhibitors; thrombocytopenic agents; and vascular repairing agents.

These and other aspects of the present invention will become apparent to those skilled in the art after a reading of the following description of the preferred embodiment when considered with the drawings.

### BRIEF DESCRIPTION OF THE DRAWINGS

The description of the features of the present invention will be more fully appreciated by reference to the following detailed description of the exemplary embodiments in accordance with the accompanying drawings, wherein:

FIG. 1 is a schematic representation of a multi-step method, showing the use of anticellular agent-carrying blood platelets, which are targeted and attached to a tumor vasculature, to treat a mammal suffering from a solid tumor, according to the present invention.

FIG. 2 is a schematic representation of another multi-step method, showing the use of anticellular agent-carrying blood platelets, which are targeted and attached to a tumor vasculature, to treat a mammal suffering from a solid tumor, according to the present invention.

FIG. 3 is a schematic representation of another multi-step method, showing the use of anticellular agent-carrying blood platelets, which are targeted and attached to a tumor vasculature, to treat a mammal suffering from a solid tumor, according to the present invention.

FIG. 4 is a schematic representation of the sequence with which the cells of a solid tumor are destroyed, on targeting anticellular agent-carrying platelets to the tumor vasculature, according to the present invention.

### DETAILED DESCRIPTION OF THE INVENTION

Prior art made of record includes the inventor's earlier U.S. patent application Ser. Nos. 11/343,694 and 11/399,281, which provides a method for treating a vascularized solid tumor by targeting a plurality of, in vitro prepared, anticellular agent-carrying blood platelets to the vasculature of the tumor, to induce a thrombus formation within the tumor vasculature, and at the same time to deliver a high concentration of the anticellular agent to the tumor cells.

However, as these before mentioned patent applications don't provide a definite mechanism with which anti-tumor antibodies may access the tumor cells, and also don't provide a definite mechanism with which the anticellular agent carried by the blood platelets may be provided to the peripherally located tumor cells, so, there exists a need for a method of treating solid tumors having a definite mechanism with which anti-tumor antibodies may access the tumor cells, and having a definite mechanism with which the anticellular agent carried by the blood platelets may be provided to the peripherally located tumor cells.

Also, as these before mentioned patent applications don't provide a definite mechanism with which relatively small sized, not yet vascularized, solid tumor metastases deposits may be targeted, so, there exists a need for a method of treating solid tumors having a definite mechanism with which small sized, not yet vascularized, solid tumor metastases deposits may be targeted.

Accordingly, the present invention is directed to and provides, in one aspect of the invention, a method for treating a vascularized solid tumor using a plurality of anticellular agent-carrying blood platelets targeted to the vasculature of the tumor, to induce a thrombus formation within the tumor vasculature and, at the same time, to deliver a high concentration of the anticellular agent to the peripherally located tumor cells, and thus, clearing solid tumors entirely from a mammalian body.

The present invention is further directed, in another aspect of the invention, to means for targeting a plurality of anticellular agent-carrying blood platelets to a tumor vasculature, to induce a thrombus formation within the tumor vasculature and, at the same time, to deliver a high concentration of the anticellular agent to the peripherally located tumor cells, while avoiding any deterioration in the functions of the spleen or any other vital body organ.

The present invention is further directed, in another aspect of the invention, to a schedule for treating a mammal affected by multi-focal secondary metastases deposits of a solid tumor, with at least one of the secondary metastases deposits being vascularized, and with at least another one of the secondary metastases deposits being not-yet vascularized.

As used herein, the term “parenteral administration” refers to and includes any route through which a compound is administered to a mammal other than through the digestive tract, non limiting examples of such routes include: intravenous injection, intra-arterial injection, intracavitary injection, intramuscular injection, and injection through an intravenous line, cannula, catheter, or the like; the term “parenteral administration of a sub-therapeutic dose of a small molecular Vascular Disrupting Agent (VDA)” refers to and includes the administration of any small molecular Vascular Disrupting Agent (VDA), to a mammal, at any dose smaller than its minimal therapeutically effective dose, with the minimal therapeutically effective dose of a VDA being the dose at or above which the administration of the VDA to the mammal will result in acute irreversible occlusion of any tumor vasculature; the term “anti-tumor binding component” refers to and includes any compound having a binding region specifically binding to an antigen or a receptor present on the outer surface of a tumor cell, or present on the outer surface of a component of a tumor associated vasculature or stroma; the terms “first ligand, second ligand, and anti-ligand” refers to and includes any complementary set of molecules that specifically bind to each other; the term “anti-platelet binding component” refers to and includes any compound having a binding region specifically binding to an antigen or a receptor present on the outer surface of a blood platelet; the term “carrying” refers to and includes either containing the anticellular agent within the blood platelets, attaching the anticellular agent to the outer surface of the blood platelet, or both; and the term “anticellular agent” refers to and includes any agent that destroys, impair the growth or cell division, or irreversibly alter the metabolism of a cancer cell.

Typical vascularized solid tumors are solid tumors which require a vascular component for the provision of oxygen and nutrients. Exemplary solid tumors to which the present invention is directed, include, but are not limited to, carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, neuroblastomas, different types of sarcomas, and the like.

Accordingly, the present invention provides method and means for treating a mammal from a vascularized solid tumor using a number of, in vitro prepared, anticellular agent-carrying blood platelets to induce a thrombus formation within the tumor vasculature, and at the same time to deliver a high concentration of the anticellular agent to the peripherally located tumor cells.

In the method provided in the present invention, a sub-therapeutic dose of a small molecular Vascular Disrupting Agent (VDA) is used to selectively disrupt the endothelial lining of the tumor vasculature, while maintaining adequate rate of blood flow within the tumor vasculature. Although small molecular VDAs are tested and developed to provide acute irreversible occlusion of the tumor vasculature, yet conducted preclinical studies showed that the administration of a low sub-therapeutic dose of a small molecular VDA results in disruption of the endothelial lining of the tumor vasculature, with initial marked reduction in the tumor blood flow, followed by near complete recovery of the blood flow within the tumor vasculature after 24 hours. A non limiting example of these studies is disclosed by Prise V E, Honess D J, Stratford M R L, Wilson J, Tozer G M., in “The vascular response of tumor and normal tissues in the rat to the vascular targeting agent, combretastatin A4-phosphate, at clinically relevant doses”. (Int J Oncol 2002; 21:717-26.). This study is incorporated herein by reference.

In a preferred embodiment of the present invention, the provided method for treating a vascularized solid tumor includes the steps of:
a) parenteral administration of a sub-therapeutic dose of at least one small molecular Vascular Disrupting Agent (VDA), to disrupt the endothelial lining of the tumor vasculature, while maintaining adequate rate of blood flow within the tumor vasculature, and thus directly exposing the underlying tumor cells to the circulating blood;
b) Targeting a number of in vitro prepared, anticellular agent-carrying, blood platelets to the tumor cells exposed in step (a), leading to immobilization of the said anticellular agent-carrying blood platelets within the tumor vasculature;
c) allowing for the induction of a thrombus formation within the tumor vasculature, leading to occlusion of the tumor vasculature and destruction of the centrally located tumor cells, followed by rupture of the said anticellular agent-carrying blood platelets, with release of their anticellular agent content; and
d) arranging for the delivery of the anticellular agent released from the ruptured blood platelets in step (c), to the peripherally located tumor cells.

In another preferred embodiment of the present invention, the provided method for treating a vascularized solid tumor includes the steps of:
a) parenteral administration of a sub-therapeutic dose of at least one small molecular Vascular Disrupting Agent (VDA), to disrupt the endothelial lining of the tumor vasculature, while maintaining adequate rate of blood flow within the tumor vasculature, and thus directly exposing the underlying tumor cells to the circulating blood. In a preferred embodiment, this step is preceded and/or accompanied by the administration of at least one anti-coagulant agent, e.g. Heparin, to prevent the formation of any small thrombi within the tumor vasculature, which is known to accompany the administration of VDA;
b) parenteral administration of a number of at least one type of anti-tumor binding component—first ligand complexes, which will attach themselves to the exposed tumor cells. In a preferred embodiment, this step is also preceded and/or accompanied by the administration of at least one anti-coagulant agent, e.g. Heparin, to prevent the formation of any small thrombi within the tumor vasculature;
c) parenteral administration of a number of anti-ligands, which will attach themselves to the anti-tumor binding component—first ligand complexes, already attached to the tumor cells. In a preferred embodiment, this step is also preceded and/or accompanied by the administration of at least one anti-coagulant agent, e.g. Heparin, to prevent the formation of any small thrombi within the tumor vasculature;
d) parenteral administration of a number of, in vitro prepared, blood platelets, each blood platelet carrying at least one anticellular agent and having at least one anti-platelet binding component—second ligand complex attached to its outer surface. In a preferred embodiment, this step is also preceded and/or accompanied by the administration of at least one anti-coagulant agent, e.g. Heparin, to delay the onset of the self-induced thrombus formation within the tumor vasculature, as will be described herein after;
e) allowing the blood platelets to link to the tumor cells through in vivo formation of anti-tumor binding component—first ligand—anti-ligand—second ligand—anti-platelet binding component complexes;
f) parenteral administration of a number of anti-ligands to allow more anticellular agent-carrying blood platelets to link to the blood platelets already linked to the tumor cells, through in vivo formation of anti-platelet binding component—second ligand—anti-ligand—second ligand—anti-platelet binding component complexes. In a preferred embodiment, this step is also preceded and/or accompanied by the administration of at least one anti-coagulant agent, e.g. Heparin, to delay the onset of the self-induced thrombus formation within the tumor vasculature, as will be described herein after, until most of the administered anticellular agent-carrying blood platelets are immobilized within the tumor vasculature;
g) allowing for the initiation of the self-induced thrombus formation within the tumor vasculature, as will be described herein after, leading to occlusion of the tumor vasculature and destruction of the centrally located tumor cells, followed by rupture of the anticellular agent-carrying blood platelets included within the formed blood thrombus, with release of their anticellular agent content; and
h) encouraging the mammal to exercise few times a day, for several days, to evenly disperse the anticellular agent released from the ruptured blood platelets within the debris of the centrally located tumor cells, which enables delivering the anticellular agent to the peripherally located tumor cells, as will be described herein after.

In a preferred embodiment, the formation of a blood thrombus within the tumor vasculature in step (g) is followed by removal of the freely circulating residual portion of the administered anticellular agent-carrying blood platelets, which were not included within the thrombus formed within the tumor vasculature, from the mammal's blood stream.

Non limiting examples of small molecular Vascular Disrupting Agent (VDA) for use with the present invention include the microtubulin destabilizing drugs, combretastatin A4 disodium phosphate, ZD6126, AVE8062, and Oxi 4503; and the flavonoid, DMXAA. Such small molecular VDAs and methods for their preparation are well known to people experienced in the Art.

Any compound having a binding region specifically binding to an antigen or a receptor present on the outer surface of a tumor cell, or present on the outer surface of a component of a tumor associated vasculature or stroma can be used as the anti-tumor binding component, according to the present invention. In a preferred embodiment of the present invention, at least two types of anti-tumor binding components are used, with one of them specifically binding to tumor cells, and the other one specifically binding to a tumor associated vasculature or stroma. Non limiting examples of anti-tumor binding components for use with the present invention include: an antibody, a monoclonal antibody, a polyclonal antibody, a humanized monoclonal antibody, a chimeric antibody, a single chain antibody, a dimeric single chain antibody construct, a multimeric single chain antibody construct, a peptide, a nucleic acid sequence, a protein, a ligand or anti-ligand, an oligonucleotide, native or naked antibodies; chimeric monoclonal antibodies; genetically engineered monoclonal antibodies; fragments of antibodies, tumor-binding peptides; polypeptide; glycoprotein; lipoprotein, growth factors; lymphokines and cytokines; enzymes, immune modulators; fusion protein, enzymatic substrate, receptor, hormone, lectin, cadherin, immunological conjugates, chemical conjugates, any of the above joined to a molecule that mediates an effector function; conjugates that include any one of the above, and fragments or parts of any of the above. Such anti-tumor binding components and methods for their preparation are well known to people experienced in the Art.

Any complementary set of molecules that specifically bind to each other can be used as the first ligand, second ligand, and anti-ligand according to the present invention. Non limiting examples of such complementary sets of molecules for use with the present invention include: biotin/avidin or streptavidin or a chemically modified form of streptavidin or avidin, zinc finger protein/dsDNA fragment, enzyme/inhibitor, hapten/antibody, ligand/receptor, homophylic peptides and leucine zipper sets. Such complementary sets of molecules and methods for their preparation are well known to people experienced in the Art. See, generally, P. Webber et al., “Science, vol. 243, pp. 85-88, Jan. 6, 1989”, M. Wilchek et al, “Analytical Biochemistry, vol. 171 pp. 1-32, 1988”, Bayer et al., “Trends in Biochemical Science, 3, N257, November 1978”, and Paganelli G, Riva P, Deleide G, et al. “Int J Cancer Suppl. 1988; 2: 121-125”; all of which are incorporated herein by reference.

Also, any compound having a binding region specifically binding to an antigen or a receptor present on the outer surface of a blood platelet can be used as the anti-platelet binding component according to the present invention. Any compound having a binding region specifically binding to an antigen or a receptor present on the outer surface of a blood platelet can be used as the anti-platelet binding component according to the present invention. Non limiting examples of anti-platelet binding components for use with the present invention include: anti-platelet monoclonal antibodies described by Gralnick in U.S. Pat. No. 5,366,865, von Willebrand factor, osteopontin, fibrinogen, fibrin, fibronectin, vitronectin, collagen, thrombospondin, laminin, heparin, heparan sulfate, chondroitin sulfate, phospholipase A2, matrix metalloproteinases, thrombin, glass, sialyl-lewis X, fibulin-1, PECAM, ICAM-1, ICAM-2, p-selectin ligand, MAC-1, LFA-1, portions of any of the above, and functional equivalents of any of the above. Such anti-platelet binding components and methods for their preparation are well known to people experienced in the Art.

“Platelets” utilized in carrying out the present invention are, in general, of animal, and preferably mammalian, origin (e.g., pig, sheep, cow, horse, goat, cat, dog, mouse, rat, human, etc.). Platelets may be derived from the same species into which the platelets are introduced, or from a species different from the species into which the platelets are introduced. In a preferred embodiment, platelets are harvested from a subject, prepared according to the method provided in the present invention, and after being so prepared are administered at a later time back to the same subject from which the platelets were harvested.

Either freshly isolated platelets or rehydrated fixed-dried platelets can be used with the present invention. In a preferred embodiment, platelets are freshly isolated, prepared according to the method provided in the present invention, and then administered, either to the same subject from whom it was harvested, or to another subject.

In another preferred embodiment, fixed-dried platelets are used, with the anticellular agent(s) and the anti-platelet binding component—second ligand complexes being attached and/or internalized into the platelets either before or after fixing and/or drying the platelets, and on a later time the platelets are rehydrated and administered either to the same subject from whom it was harvested, or to another subject. The use of fixed-dried platelets enables safe extension of the time period between the harvesting of the platelets and their administering. Platelets may be fixed in accordance with known techniques, such as described in U.S. Pat. Nos. 4,287,087; 5,651,966; 5,902,608; 5,891,393; and 5,993,084. Drying of platelets after fixation may be carried out by any suitable means, such as lyophilization.

Any agent that destroys, impairs the growth or cell division, or irreversibly alters the metabolism of cancer cells can be used as the anticellular agent, according to the present invention. Non limiting examples of anti-cellular agents for use with the present invention include: cytotoxins, chemotherapeutic agents; steroids, antimetabolites, anthracyclines, vinca alkaloids, antibiotics, alkylating agents, epipodophyllotoxins; any plant-, fungus- or bacteria-derived agent; and radioactive isotopes such as ¹²⁵I, ¹³¹I, and ⁸⁶Rb,

In a preferred embodiment of the present invention, the anticellular agent(s) is internalized into the blood platelets, using any suitable technique. Non limiting examples of the techniques with which an anticellular agent may be internalized into the platelets are: (1) conjugating the compound to be delivered to a polymer that is in turn coupled to the platelet's internal membrane; (2) incorporating the compound to be delivered into unilamellar or multilamellar phospholipid vesicles that are in turn internalized into the platelets; (3) absorbing or internalizing the compound to be delivered into nanoparticles, e.g., buckminsterfullerene, that are in turn internalized into the platelets; (4) coupling the compound to be delivered to proteins that are internalized for trafficking to alpha granules in the platelets; (5) coupling the compound to be delivered to proteins (or other macromolecules) or particles that are phagocitized by the platelets; (6) adsorbing the compound to the exterior surface of the cell by non-covalent physical or chemical adsorption, that are in turn internalized into the platelets; and (7) physically entrapping the compound to be delivered in the platelet intracellular space through pores that are formed with electroporation, complement treatment, lytic protein exposure, and the like. These and other techniques used for intraluminal delivery of drugs are well known to people experienced in the Art.

In another preferred embodiment, the anticellular agent(s) is attached to the outer surface of the blood platelet, using any suitable technique. Non limiting examples of the techniques with which an anticellular agent may be attached to the outer surface of the platelets are: (1) directly chemically coupling the compound to be delivered to the platelet surface membrane; (2) attaching the anticellular agent to an anti-platelet binding component, e.g. an antibody or a ligand, which attaches to an antigen or a receptor on the outer surface of the blood platelets; and (3) adsorbing the compound to the exterior surface of the cell by non-covalent physical or chemical adsorption. These and other techniques used for attaching a compound to the outer surface of platelets or cells are well known to people experienced in the Art.

In yet another preferred embodiment, more than one anticellular agent are used, with at least one anticellular agent being internalized into the blood platelet, and at least one anticellular agent being attached to the outer surface of the blood platelet, using any combination of the techniques described herein above.

In general, the anticellular agent(s) to be delivered is coupled to or associated with the platelets so that each platelet carries, or has associated therewith, at least 1,000, and more preferably at least 10,000, individual molecules of the agent to be delivered.

As the life span of the platelets within the formed thrombus is approximately 10 days, so, everyday about 10% of the platelets attached to the cancer cells, or to the tumor associated vasculature or stroma, will rupture spontaneously. The ruptured platelets will release ADP (Adenosine diphosphate), thromboxane A2, serotonin, phospholipids, lipoproteins, and other proteins, leading to the activation of the nearby blood platelets and the initiation of a blood coagulation cascade. See, for example: Hechler, B., Leon, C., Vial, C., Vigne, P., Frelin, C., Cazenave, J. P., and Gachet, C. (1998) Blood 92, 152-159. The activation of the blood platelets modifies their membranes in such a way to allow fibrinogen to adhere to them, which results in attaching the fibrinogen net of the formed blood thrombus to the outer surface of the activated blood platelets. And thus, the formed blood thrombus will be indirectly attached to the tumor cells.

The formed blood thrombus occludes the blood vessels in-between the cancer cells, and thus, cutting off the blood supply to the centrally located cancer cells, leading to their destruction. This is followed by rupture of the platelets immobilized at the periphery of the formed blood thrombus, with release of their anticellular agent content. Then, the patient is encouraged to exercise few times a day, for several days, to mechanically agitate the cellular debris within the central part of the tumor, leading to even dispersion of the anticellular agent, released from the ruptured blood platelets, within the debris of the centrally located tumor cells, and thus enabling the anticellular agent to reach to the intact peripherally located tumor cells at high concentration, leading to their subsequent destruction; or impairment of their growth or cellular division; or irreversibly altering their metabolism, according to the type of the anticellular agent(s) used, and thus, all the cells of the solid tumor are destroyed.

The freely circulating residual portion of the administered anticellular agent-carrying blood platelets, which were not included within the thrombus formed within the tumor vasculature, is non-selectively removed from the mammal's blood stream using the well known apheresis procedure. In another preferred embodiment, the freely circulating residual portion of the administered anticellular agent-carrying blood platelets, which were not included within the thrombus formed within the tumor vasculature, is selectively removed from the mammal's blood stream using sheet membranes, hollow fibers, or packed beds of either beads or particles having physically adsorbed or covalently attached anti-ligands. The anti-ligands will selectively bind the anticellular agent-carrying blood platelets through the formation anti-ligand—second ligand—anti-platelet binding component complexes, and thus, selectively removing the freely circulating residual portion of the administered blood platelets from the mammal's blood stream. Such techniques and means for their conduction are well known to people experienced in the Art.

In a preferred embodiment of the present invention, the provided method is preceded and/or accompanied and/or followed by conventional enteral or parenteral administration of a therapeutic dose of at least one anticellular agent, to destroy any small sized non vascularized tumors present within the mammal, as well as early implanted and not-yet implanted tumor metastases.

Also, in a preferred embodiment of the present invention, the provided method is preceded and/or accompanied by the administration of at least one immuno-suppressive agent to the mammal, to safe guard against the development of an immune response against the administered components which will hinder their re-administration in a following setting, if needed. Non-limiting examples of immunosuppressive agents for use with the present invention includes: alkylating agents such as cyclophosamide; nucleic acid antimetabolites such as 6-mercaptopurine and azathiopurine; antibiotics such as mitomycin C; steroids; folic acid antagonists such as methotrexate; and plant alkaloids such as colchicine and vinblastine; and cyclic polypeptides such as cyclosporine. Such immunosuppressive agents are well known to people experienced in the Art.

The present invention is further directed, in another aspect of the invention, to a schedule for treating a mammal affected by multi-focal, variable-sized, secondary metastases deposits of a solid tumor, with at least one of the secondary metastases deposits being vascularized, and with at least another one of the secondary metastases deposits being not-yet vascularized.

In a preferred embodiment, the provided schedule for treating the mammal affected by said multi-focal, variable-sized, secondary metastases deposits of the solid tumor includes the steps of:
a) treating the mammal using the method provided in the present invention for treating a mammal from a vascularized solid tumor, and described in full details herein before, to destroy the vascularized secondary metastases deposits of the tumor;
b) administration of at least one agent to prevent/minimize the implantation of new tumor secondary metastases deposits within the mammal's body, for a period of time sufficient for the not-yet vascularized secondary metastases deposits of the tumor to grow and develop its own vasculature; and
c) re-treating the mammal using the method provided in the present invention for treating a mammal from a vascularized solid tumor, to destroy the now vascularized secondary metastases deposit(s) of the tumor, and thus, completely clearing the secondary metastases deposits of the tumor from the mammal's body.

In a preferred embodiment, the agent(s) administered to prevent/minimize the implantation of new tumor secondary metastases deposits within the mammal's body is selected from the group consisting of anti-coagulants, e.g. Vitamin K antagonists such as Dicumarol, Warfarin, or others; platelet inhibitors; thrombocytopenic agents; and vascular repairing agents. The role of such agents in minimizing/preventing the implantation of new tumor secondary metastases deposits within a mammal's body has been confirmed in different studies conducted to explore the role of blood platelets in tumor metastases. A non limiting example of these studies is disclosed by Gabriel J. Gasic in “Role of plasma, platelets, and endothelial cells in tumor metastases” (Cancer and Metastases Reviews, volume 3, Number 2/June 1984: 99-114). This study is incorporated herein by reference.

These and other aspects of the present invention will become apparent to those skilled in the art after a reading of the following description of the preferred embodiment when considered with the drawings.

In the following preferred embodiments the “anti-tumor binding component—first ligand complex” is exemplified by a “biotinylated anti-tumor antibody”; the “anti-ligand” is exemplified by “avidin, or avidin like molecules”; and the “anti-platelet binding component—second ligand complex” is exemplified by a “biotinylated anti-platelet antibody”, with freshly isolated blood platelets being used as the vehicle through which the anticellular agents are being delivered. These components and techniques are used as illustrative examples, and are not intended to limit the scope of the compounds and techniques that may be used according to the present invention.

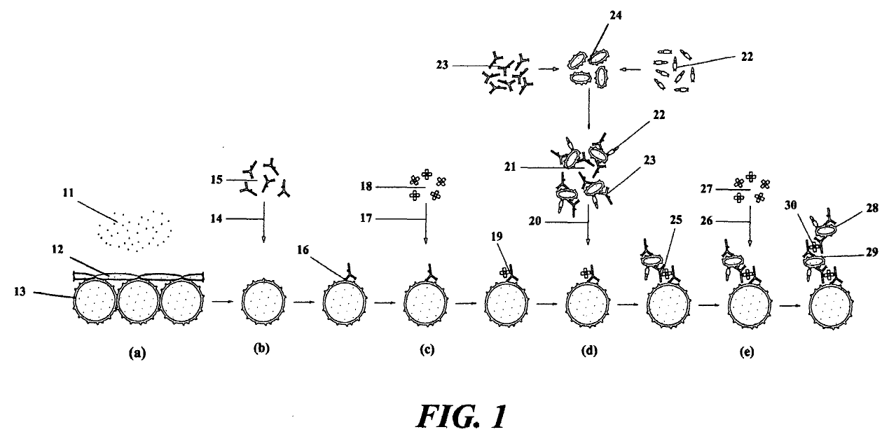

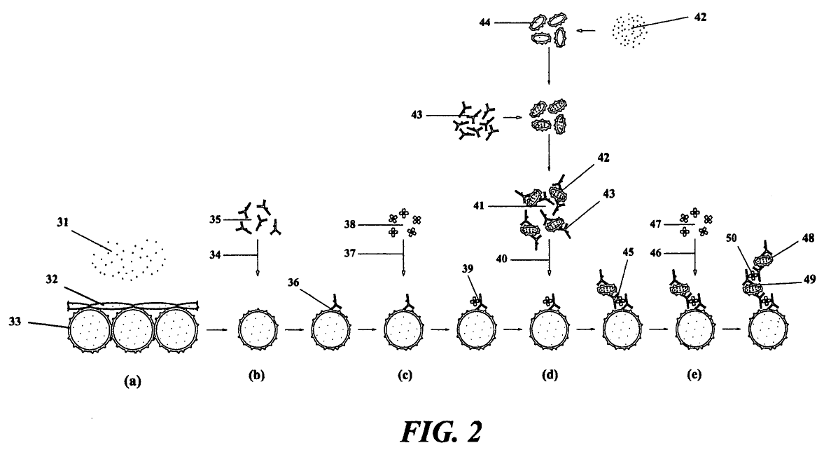

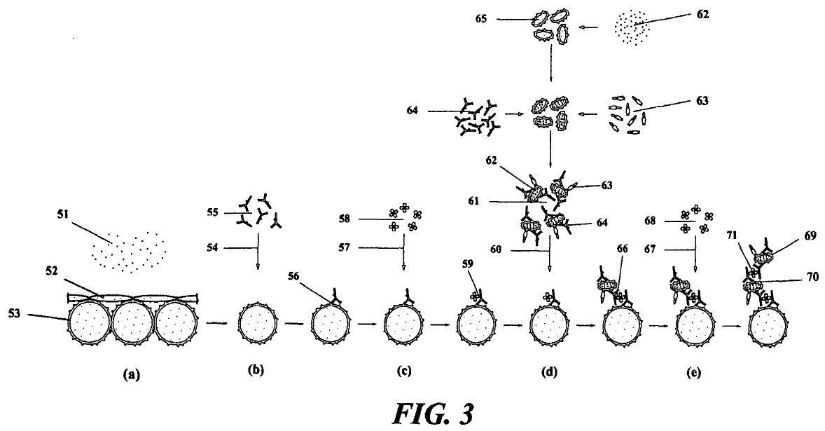

FIG. 1 is a schematic representation of a multi-step method, showing the use of anticellular agent-carrying blood platelets, which are targeted and attached to a tumor vasculature, to treat a mammal suffering from a solid tumor, according to the present invention.
The provided method includes the steps of:
a) parenteral administration of a sub-therapeutic dose of a small molecular Vascular Disrupting Agent (VDA) (11), to disrupt the endothelial lining (12) of the tumor vasculature, while maintaining adequate rate of blood flow within the tumor vasculature, and thus directly exposing the underlying tumor cells (13) to the circulating blood;
b) parenteral administration (14) of a number of biotinylated anti-tumor antibodies (15), which will attach themselves to the tumor (16);
c) parenteral administration (17) of a number of avidin or avidin like molecules (18), which will attach themselves (19) to the biotinylated anti-tumor antibodies already attached to the tumor, along with attaching any free circulating non implanted tumor metastases to the main tumor bulk (not shown in the drawing for simplicity);
d) parenteral administration (20) of a number of, in vitro prepared, blood platelets (21), each blood platelet has at least one anticellular agent (22) and at least one biotinylated anti-platelet antibody (23) attached to its outer surface. The in vitro preparation of the administered blood platelets includes the steps of: collecting a number of blood platelets (24), either from the same mammal or from an immunologically compatible mammal using, for example, the well known apheresis procedure; attaching the used anticellular agent (22) to the outer surface of the platelets using one of the techniques described herein above; and incubating the collected blood platelets in a solution having biotinylated anti-platelet antibodies (23) within it for a time sufficient for the complexes to attach to the outer surface of the platelets (21);
e) allowing the blood platelets to link to the tumor vasculature through in vivo formation of biotin-avidin-biotin or biotin-avidin like-biotin linkages (25) with the biotinylated anti-tumor antibodies already attached to the tumor;
f) parenteral administration (26) of a number avidin or avidin like molecules (27), to allow more blood platelets (28) to link to the blood platelets (29) already linked to the tumor vasculature through in vivo formation of biotin-avidin-biotin or biotin-avidin like-biotin linkages (30) with the biotinylated anti-platelet antibodies attached to the surfaces of the platelets already attached to the tumor, with this step being accompanied by The administration of at least one anti-coagulant agent, e.g. Heparin, to delay the onset of the self-induced thrombus formation within the tumor vasculature, as will be described herein before, until most of the administered anticellular agent-carrying blood platelets are immobilized within the tumor vasculature;
g) stopping the anti-coagulant administration to allow for the initiation of the self-induced thrombus formation within the tumor vasculature, as will be described herein before;
h) removing the freely circulating residual portion of the administered anticellular agent-carrying blood platelets, which were not included within the thrombus formed within the tumor vasculature, from the mammal's blood stream. This step is not shown in the drawing for simplicity.
i) encouraging the mammal to exercise few times a day, for several days, to evenly disperse the anticellular agent released from the ruptured blood platelets within the debris of the centrally located tumor cells, which enables delivering the anticellular agent to the peripherally located tumor cells, as will be described herein after.
   FIG. 2 is a schematic representation of another multi-step method, showing the use of anticellular agent-carrying blood platelets, which are targeted and attached to the tumor vasculature, to treat a mammal suffering from a solid tumor, according to the present invention.
   The provided method includes the steps of:
a) parenteral administration of a sub-therapeutic dose of a small molecular Vascular Disrupting Agent (VDA) (31), to disrupt the endothelial lining (32) of the tumor vasculature, while maintaining adequate rate of blood flow within the tumor vasculature, and thus directly exposing the underlying tumor cells (33) to the circulating blood;
b) parenteral administration (34) of a number of biotinylated anti-tumor antibodies (35), which will attach themselves to the tumor (36);
c) parenteral administration (37) of a number of avidin or avidin like molecules (38), which will attach themselves (39) to the biotinylated anti-tumor antibodies already attached to the tumor, along with attaching any free circulating non implanted tumor metastases to the main tumor bulk (not shown in the drawing for simplicity);
d) parenteral administration (40) of a number of, in vitro prepared, blood platelets (41), each blood platelet has at least one anticellular agent (42) and at least one biotinylated anti-platelet antibody (43) attached to its outer surface. The in vitro preparation of the administered blood platelets includes the steps of: collecting a number of blood platelets (44), either from the same mammal or from an immunologically compatible mammal using, for example, the well known apheresis procedure; internalizing the used anticellular agent (42) inside the blood platelets using one of the techniques described herein above; and incubating the collected blood platelets in a solution having biotinylated anti-platelet antibodies (43) within it for a time sufficient for the complexes to attach to the outer surface of the platelets (41);
e) allowing the blood platelets to link to the tumor vasculature through in vivo formation of biotin-avidin-biotin or biotin-avidin like-biotin linkages (45) with the biotinylated anti-tumor antibodies already attached to the tumor;
f) parenteral administration (46) of a number avidin or avidin like molecules (47), to allow more blood platelets (48) to link to the blood platelets (49) already linked to the tumor vasculature through in vivo formation of biotin-avidin-biotin or biotin-avidin like-biotin linkages (50) with the biotinylated anti-platelet antibodies attached to the surfaces of the platelets already attached to the tumor, with this step being accompanied by The administration of at least one anti-coagulant agent, e.g. Heparin, to delay the onset of the self-induced thrombus formation within the tumor vasculature, as will be described herein before, until most of the administered anticellular agent-carrying blood platelets are immobilized within the tumor vasculature;
g) stopping the anti-coagulant administration to allow for the initiation of the self-induced thrombus formation within the tumor vasculature, as will be described herein before;
h) removing the freely circulating residual portion of the administered anticellular agent-carrying blood platelets, which were not included within the thrombus formed within the tumor vasculature, from the mammal's blood stream. This step is not shown in the drawing for simplicity.
i) encouraging the mammal to exercise few times a day, for several days, to evenly disperse the anticellular agent released from the ruptured blood platelets within the debris of the centrally located tumor cells, which enables delivering the anticellular agent to the peripherally located tumor cells, as will be described herein after.
   FIG. 3 is a schematic representation of another multi-step method, showing the use of anticellular agent-carrying blood platelets, which are targeted and attached to the tumor vasculature, to treat a mammal suffering from a solid tumor, according to the present invention.
   The provided method includes the steps of:
a) parenteral administration of a sub-therapeutic dose of a small molecular Vascular Disrupting Agent (VDA) (51), to disrupt the endothelial lining (52) of the tumor vasculature, while maintaining adequate rate of blood flow within the tumor vasculature, and thus directly exposing the underlying tumor cells (53) to the circulating blood;
b) parenteral administration (54) of a number of biotinylated anti-tumor antibodies (55), which will attach themselves to the tumor (56);
c) parenteral administration (57) of a number of avidin or avidin like molecules (58), which will attach themselves (59) to the biotinylated anti-tumor antibodies already attached to the tumor, along with attaching any free circulating non implanted tumor metastases to the main tumor bulk (not shown in the drawing for simplicity);
d) parenteral administration (60) of a number of, in vitro prepared, blood platelets (61), each blood platelet has at least one anticellular agent within its cavity (62), and at least one anticellular agent (63) and at least one biotinylated anti-platelet antibody (64) attached to its outer surface. The in vitro preparation of the administered blood platelets includes the steps of: collecting a number of blood platelets (65), either from the same mammal or from an immunologically compatible mammal using, for example, the well known apheresis procedure; internalizing one of the used anticellular agents (62) inside the blood platelets and attaching the other anticellular agent (63) to the outer surface of the platelets using any combination of the techniques described herein above; and incubating the collected blood platelets in a solution having biotinylated anti-platelet antibodies (64) within it for a time sufficient for the complexes to attach to the outer surface of the platelets (61);
e) allowing the blood platelets to link to the tumor vasculature through in vivo formation of biotin-avidin-biotin or biotin-avidin like-biotin linkages (66) with the biotinylated anti-tumor antibodies already attached to the tumor;
f) parenteral administration (67) of a number avidin or avidin like molecules (68), to allow more blood platelets (69) to link to the blood platelets (70) already linked to the tumor vasculature through in vivo formation of biotin-avidin-biotin or biotin-avidin like-biotin linkages (71) with the biotinylated anti-platelet antibodies attached to the surfaces of the platelets already attached to the tumor, with this step being accompanied by The administration of at least one anti-coagulant agent, e.g. Heparin, to delay the onset of the self-induced thrombus formation within the tumor vasculature, as will be described herein before, until most of the administered anticellular agent-carrying blood platelets are immobilized within the tumor vasculature;
g) stopping the anti-coagulant administration to allow for the initiation of the self-induced thrombus formation within the tumor vasculature, as will be described herein before;
h) removing the freely circulating residual portion of the administered anticellular agent-carrying blood platelets, which were not included within the thrombus formed within the tumor vasculature, from the mammal's blood stream. This step is not shown in the drawing for simplicity.
i) encouraging the mammal to exercise few times a day, for several days, to evenly disperse the anticellular agent released from the ruptured blood platelets within the debris of the centrally located tumor cells, which enables delivering the anticellular agent to the peripherally located tumor cells, as will be described herein after.

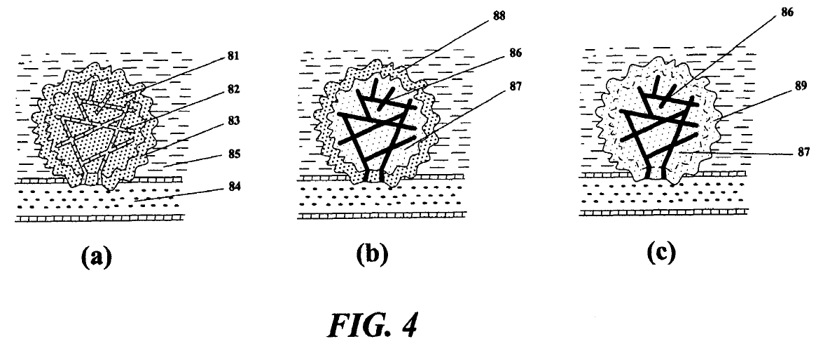

### FIG. 4 is a schematic representation of the sequence with which the cells of a solid tumor are destroyed, on targeting anticellular agent-carrying platelets to the tumor vasculature, according to the present invention.

For illustrative purposes, as shown in step (a), the tumor cells are divided into two portions: a first portion comprising centrally located tumor cells (81), which depends for their nutrition on the tumor vasculature (82); and a second portion comprising peripherally located tumor cells (83), which depend for their nutrition on the surrounding blood vessels (84) and surrounding interstitial fluid (85).

The platelet-mediated thrombus formed within the tumor vasculature (86) leads to occlusion of the tumor vasculature (82), with ultimate destruction of the centrally located tumor cells (87), and as shown in step (b). This is followed by rupture of the anticellular agent-carrying blood platelets immobilized at the periphery of the formed blood thrombus (86), with release of their anticellular agent content. Then, the patient is encouraged to exercise few times a day, for several days, to mechanically agitate the cellular debris within the central part of the tumor, leading to even dispersion of the anticellular agent, released from the ruptured blood platelets, within the debris of the centrally located tumor cells (87), and thus enabling the anticellular agent to reach to the intact peripherally located tumor cells (88) at high concentration, leading to their subsequent destruction (89); or impairment of their growth or cellular division; or irreversibly altering their metabolism, according to the type of the anticellular agent(s) used, as shown in step (c), and thus, all the cells of the solid tumor are destroyed.

Certain modifications and improvements will occur to those skilled in the art upon a reading of the detailed description. All modifications and improvements have been deleted herein for the sake of conciseness and readability but are properly within the scope of the following claims.

What is claimed is:

1. In a mammal, a method for treating a vascularized solid tumor, including the steps of:
a) parenteral administration of a sub-therapeutic dose of at least one small molecular Vascular Disrupting Agent (VDA), to disrupt the endothelial lining of the tumor vasculature, while maintaining adequate rate of blood flow within the tumor vasculature, and thus directly exposing the underlying tumor cells to the circulating blood;
b) Targeting a number of, in vitro prepared, anticellular agent-carrying blood platelets to the tumor cells exposed in step (a), leading to immobilization of the said anticellular agent-carrying blood platelets within the tumor vasculature;
c) allowing for the induction of a thrombus formation within the tumor vasculature, leading to occlusion of the tumor vasculature and destruction of the centrally located tumor cells, followed by rupture of the said anticellular agent-carrying blood platelets, with release of their anticellular agent content; and
d) arranging for the delivery of the anticellular agent released from the ruptured blood platelets in step (c), to the peripherally located tumor cells.

2. In a mammal, a method for treating a vascularized solid tumor, including the steps of:
a) parenteral administration of a sub-therapeutic dose of at least one small molecular Vascular Disrupting Agent (VDA), to disrupt the endothelial lining of the tumor vasculature, while maintaining adequate rate of blood flow within the tumor vasculature, and thus directly exposing the underlying tumor cells to the circulating blood;
b) parenteral administration of a number of at least one type of anti-tumor binding component—first ligand complexes;
c) parenteral administration of a number of anti-ligands;
d) parenteral administration of a number of, in vitro prepared, blood platelets, each blood platelet carrying at least one anticellular agent and having at least one anti-platelet binding component—second ligand complex attached to its outer surface;
e) allowing the blood platelets to link to the tumor vasculature through in vivo formation of anti-tumor binding component—first ligand—anti-ligand—second ligand—anti-platelet binding component complexes;
f) parenteral administration of a number of anti-ligands;
g) allowing more anticellular agent-carrying blood platelets to link to the blood platelets already linked to the tumor vasculature through in vivo formation of anti-platelet binding component—second ligand—anti-ligand—second ligand—anti-platelet binding component complexes, thereby allowing for the formation of a blood thrombus within the tumor vasculature, leading to occlusion of the tumor vasculature and destruction of the centrally located tumor cells, followed by rupture of the anticellular agent-carrying blood platelets included within the formed blood thrombus, with release of their anticellular agent content; and
h) encouraging the mammal to exercise few times a day, for several days, thereby evenly dispersing the anticellular agent released from the ruptured blood platelets within the debris of the centrally located tumor cells, which enables delivering the anticellular agent to the peripherally located tumor cells.

3. The method of claim 2, wherein the formation of a blood thrombus within the tumor vasculature in step (g) is followed by removal of the freely circulating residual portion of the administered anticellular agent-carrying blood platelets, which were not included within the thrombus formed within the tumor vasculature, from the mammal's blood stream.

4. The method of claim 2, wherein at least one of the said steps of the method is preceded and/or accompanied by the administration of at least one anti-coagulant agent to the mammal.

5. The method of claim 2, which is preceded and/or accompanied by the administration of at least one immuno-suppressive agent to the mammal.

6. The method of claim 2, which is preceded and/or accompanied and/or followed by enteral or parenteral administration of a therapeutic dose of at least one anticellular agent.

7. The method of claim 2, wherein the small molecular Vascular Disrupting Agent (VDA) is selected from the group consisting of the microtubulin destabilizing drugs, combretastatin A-4 disodium phosphate, ZD6126, AVE8062, and Oxi 4503; and the flavonoid, DMXAA.

8. The method of claim 2, wherein the anti-tumor binding component has a binding region specifically binding to an antigen or a receptor present on the outer surface of a tumor cell, or present on the outer surface of a component of a tumor associated vasculature or stroma, with said anti-tumor binding component being selected from the group consisting of an antibody, a monoclonal antibody, a polyclonal antibody, a humanized monoclonal antibody, a chimeric antibody, a single chain antibody, a dimeric single chain antibody construct, a multimeric single chain antibody construct, a peptide, a nucleic acid sequence, a protein, a ligand or anti-ligand, an oligonucleotide, native or naked antibodies; chimeric monoclonal antibodies; genetically engineered monoclonal antibodies; fragments of antibodies, tumor-binding peptides; polypeptide; glycoprotein; lipoprotein, growth factors; lymphokines and cytokines; enzymes, immune modulators; fusion protein, enzymatic substrate, receptor, hormone, lectin, cadherin, immunological conjugates, chemical conjugates, any of the above joined to a molecule that mediates an effector function; conjugates that include any one of the above; and fragments or parts of any of the above.

9. The method of claim 2, wherein at least two types of anti-tumor binding components are used, with at least one of them specifically binding to an antigen or a receptor present on the outer surface of a tumor cell, and at least another one of them specifically binding to an antigen or a receptor present on the outer surface of a component of a tumor associated vasculature or stroma.

10. The method of claim 2, wherein the first and second ligands and the anti-ligand consist of a complementary set of molecules that specifically bind to each other and are selected from the group consisting of biotin/avidin or streptavidin or a chemically modified form of streptavidin or avidin; zinc finger protein/dsDNA fragment; enzyme/inhibitor; hapten/antibody; ligand/receptor; homophylic peptides; and leucine zipper sets.

11. The method of claim 2, wherein the anti-platelet binding component has a binding region specifically binding to an antigen or a receptor present on the outer surface of the blood platelet and is selected from the group consisting of an antibody, a monoclonal antibody, von Willebrand factor, osteopontin, fibrinogen, fibrin, fibronectin, vitronectin, collagen, thrombospondin, laminin, heparin, heparan sulfate, chondroitin sulfate, phospholipase A2, matrix metalloproteinases, thrombin, glass, sialyl-lewis X, fibulin-1, PECAM, ICAM-1, ICAM-2, p-selectin ligand, MAC-1, LFA-1, portions of any of the above, and functional equivalents of any of the above.

12. The method of claim 2, wherein the blood platelets are freshly isolated platelets.

13. The method of claim 2, wherein the blood platelets are rehydrated fixed-dried platelets.

14. The method of claim 2, wherein the anticellular agent is selected from the group consisting of radioactive isotopes, cytotoxins, chemotherapeutic agents, steroids, antimetabolites, anthracyclines, vinca alkaloids, antibiotics, alkylating agents, epipodophyllotoxins, and any plant-, fungus- or bacteria-derived agent.

15. The method of claim 2, wherein the anticellular agent is contained within the blood platelet.

16. The method of claim 2, wherein the anticellular agent is attached to the outer surface of the blood platelet.

17. The method of claim 2, wherein more than one anticellular agent are used, with at least one anticellular agent being contained within the blood platelet, and at least another anticellular agent being attached to the outer surface of the blood platelet.

18. The method of claim 2, wherein the mammal is a human cancer patient.

19. In a mammal affected by multi-focal, variable-sized, secondary metastases deposits of a solid tumor, with at least one of the secondary metastases deposits being vascularized, and at least another one of the secondary metastases deposits being not-yet vascularized, a schedule for treating said solid tumor secondary metastases deposits including the steps of:
a) treating the mammal using the method of claim 2, to destroy the vascularized secondary metastases deposit(s) of the tumor;
b) administration of at least one agent to prevent/minimize the implantation of new tumor secondary metastases deposits within the mammal's body, for a period of time sufficient for the not-yet vascularized secondary metastases deposit(s) of the tumor to grow and develop its own vasculature; and
c) re-treating the mammal using the method of claim 2, to destroy the now vascularized secondary metastases deposit(s) of the tumor, and thus, completely clearing the secondary metastases deposits of the tumor from the mammal's body.

20. The method of claim 19, wherein the administered agent(s) to prevent/minimize the implantation of new tumor secondary metastases deposits within the mammal's body is selected from the group consisting of anti-coagulants; platelet inhibitors; thrombocytopenic agents; and vascular repairing agents.

21. The method of claim 19, wherein the mammal is a human cancer patient.
